(19) United States
(12) Patent Application Publication
(10) Pub. No.: US 2026/0324972 A1
(43) Pub. Date: Oct. 22, 2026

(54) INTRAOCULAR PRESSURE SENSOR SYSTEM AND METHOD

(71) Applicant: Alcon Inc., Fribourg (CH)

(72) Inventor: Venjamin Stojanov, Dietlikon (CH)

(21) Appl. No.: 19/468,040

(22) Filed: Feb. 3, 2026

Related U.S. Application Data
(60) Provisional application No. 63/754,022, filed on Feb. 5, 2025.

(51) Int. Cl. A61B 3/16

(52) U.S. Cl. CPC A61B 3/16

(57) ABSTRACT

An intraocular pressure sensor system and method are described. The intraocular pressure sensor system includes a surface acoustic wave modifier and at least one signal receiver. The surface acoustic wave modifier modifies an initial surface acoustic wave signal based on intraocular pressure of an eye of a patient to produce a modified surface acoustic wave signal. The at least one signal receiver receives a first signal and produces the initial surface acoustic wave signal based upon the first signal. The at least one signal receiver also receives the modified surface acoustic wave signal and produces a second signal based upon the modified surface acoustic wave signal. The second signal is comparable to the first signal to determine a value indicative of the intraocular pressure of the eye.

### BACKGROUND

Measurement and/or monitoring of intraocular pressure (IOP) is important in a variety of contexts for providing proper eye care. As one example, measuring and/or monitoring of intraocular pressure can be helpful in detecting and diagnosing of eye conditions such as glaucoma. Elevated IOP can be an indicator for risk of glaucoma and can be useful in determining the severity of glaucoma. As a further example, monitoring of IOP can be helpful in determining whether a medication is adequately controlling (e.g., lowering) the IOP of a patient or if a patient is consistently taking their medication. In another context, measurements of IOP can be helpful in determining if an individual is a good candidate for a procedure such as an eye surgery. In yet another context, measurements of IOP can help determine if an eye of a patient is experiencing ocular hypotony (reduced or lower than normal IOP), which can lead to eye conditions such as hypotony maculopathy or phthisis bulbi.

Currently, multiple techniques and technologies exist for measuring IOP. As an example, an ocular tonometer is commonly used to measure IOP. For such a measurement, a contacting surface of the tonometer is pressed against the cornea of any eye thereby flattening a portion of the cornea. In turn, IOP is measured by how much pressure is exerted against the contacting surface by the flattened portion of the cornea. Measuring of IOP with a tonometer, however, has multiple drawbacks. Tonometer measurements are often uncomfortable for patients. Tonometer measurements can also be imprecise since they can vary based upon the thickness and/or rigidity of the cornea of a particular patient. Further, a tonometer is typically operated by a trained professional such that frequent and/or continuous monitoring of IOP is difficult, if not impossible, using a tonometer. Over many years, several alternative techniques and technologies have been developed to improve upon the tonometer or replace the tonometer. However, these alternative techniques and technologies for measuring IOP suffer from their own sets of drawbacks.

### SUMMARY

Intraocular pressure sensor system and method are described. The intraocular pressure sensor system includes a surface acoustic wave modifier and at least one signal receiver. The surface acoustic wave modifier modifies an initial surface acoustic wave signal based on intraocular pressure of an eye of a patient to produce a modified surface acoustic wave. The at least one signal receiver performs the following: i) receives a first signal and produces the initial surface acoustic wave signal based upon the first signal; ii) transmits the initial surface acoustic wave signal to the surface acoustic wave modifier; iii) receives the modified surface acoustic wave signal; and iv) produces a second signal based upon the modified surface acoustic wave signal. The second signal is comparable to the first signal to determine a value indicative of the intraocular pressure of the eye. As such, the intraocular pressure system can also include a device (e.g., a signal reader) for comparing the first signal to the second signal for determining the value indicative of the intraocular pressure of the eye.

The method of sensing intraocular pressure of an eye of a patient includes the following:implanting a sensor into a suprachoroidal space of the eye of the patient, the sensor including a surface acoustic wave modifier and at least one signal receiver;receiving a first signal with the at least one signal receiver, the at least one signal receiver producing an initial surface acoustic wave signal based upon the first signal;transmitting the initial surface acoustic wave signal to the surface acoustic wave modifier;modifying the initial surface acoustic wave signal using the surface acoustic wave modifier to produce a modified surface acoustic wave signal based on the intraocular pressure of the eye of the patient;transmitting the modified surface acoustic wave signal to the at least one signal receiver; and producing a second signal based upon the modified surface acoustic wave signal using the at least one signal receiver, the second signal being comparable to the first signal to determine a value indicative of the intraocular pressure of the eye.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures. Entities represented in the figures are indicative of one or more entities and thus, reference is made interchangeably to single or plural forms of the entities in the discussion.

FIG. 1 depicts a schematic diagram of an example of an intraocular pressure sensor system that is operable to determine a value indicative of intraocular pressure of an eye of a patient as described herein.

FIG. 2 depicts a flowchart of an example of a methodology for determining a value indicative of intraocular pressure of an eye of a patient as described herein.

FIG. 3A depicts a perspective view of an example of a sensor useful for determining a value indicative of intraocular pressure of an eye of a patient as described herein.

FIG. 3B depicts a sectional view of the sensor of FIG. 3A.

FIG. 4 depicts a diagram of an example implementation of an intraocular pressure sensor system that includes the sensor of FIG. 3A and 3B and a smart phone and is operable to determine a value indicative of intraocular pressure of an eye of a patient as described herein.

### DETAILED DESCRIPTION

### Overview

A healthy human eye typically has an intraocular pressure (IOP) that is within a certain range (e.g., from about 10 to about 21 millimeters of mercury (mmHg)). In contrast, a human eye having IOP outside of this range may have one or more conditions that can negatively affect that eye. Further, some individuals can have IOP within the aforementioned “normal” range, but changes in their IOP can indicate one or more conditions that can negatively affect their eye[s]. As such, measuring and/or monitoring IOP is an important part of maintaining eye health and/or addressing conditions of eyes that have or could be developing one or more negative effects in the eyes.

One quite common condition typically caused by elevated IOP is glaucoma. The elevated IOP in an individual’s eye can cause damage to the optic nerve of that eye resulting in impairment or even loss of vision in that eye. Other conditions and/or events can also cause undesirable changes (e.g., spikes) in IOP, which can be damaging to the eye. As one example, certain surgeries can result in changes to IOP. As another example, certain medications can cause undesirable IOP fluctuations.

Due to the problems that are often associated with IOP, it is quite important to have reliable and desirable techniques and tools for measuring IOP. Currently, however, techniques and tools for measuring IOP suffer from a variety of drawbacks. As one example, techniques and tools that interact with the cornea of any eye can be inaccurate since the rigidity and thickness of the cornea vary from patient to patient. As another example, certain tools and techniques are designed to be used only by trained professionals making it difficult to provide frequent monitoring or measurement of IOP. As yet another example, certain techniques and tools can cause significant discomfort to a patient.

Accordingly, an intraocular pressure sensor system is disclosed along with a methodology for sensing and/or measuring IOP of an eye of a patient using the system or parts thereof. By way of example, the intraocular pressure sensor system includes a surface acoustic wave modifier and at least one signal receiver. In operation, the at least one signal receiver receives a first signal and produces an initial surface acoustic wave signal based upon the first signal. The surface acoustic wave modifier receives the initial surface acoustic wave signal from the at least one signal receiver and modifies the initial surface acoustic wave signal based on intraocular pressure of an eye of a patient to produce a modified surface acoustic wave. The at least one signal receiver receives the modified surface acoustic wave signal and produces a second signal based upon the modified surface acoustic wave signal. For determining intraocular pressure of the eye, the second signal is compared to the first signal to determine a value indicative of the intraocular pressure of the eye.

For determining the value indicative of the intraocular pressure, the system can, according to one non-limiting aspect, include a reader that can compare the first signal sent to the at least one signal receiver to the second signal sent from the at least one signal receiver. The reader is typically capable of reading (e.g., determining frequency) the first signal and the second signal for determining a difference between the signals and correlating the difference between the signals to a value. That value is then correlated using the reader or otherwise to determine the intraocular pressure of the eye. It is contemplated that the reader can be a dedicated instrument (e.g., an electronic and/or digital instrument specifically designed for reading and/or processing the first and second signal described herein). Alternatively, the reader can be integrated into another device such as a smartphone, computer, or watch as, for example, an application of that device.

It is contemplated that the first signal and second signal can be selected from a variety of signals. According to one aspect of the present disclosure, the first signal and second signal are both radio frequency signals. Radio frequency signals may be desirable for their relative ease of reading and relative to ease of converting to and from surface acoustic wave signals.

According to an aspect of the present disclosure, the at least one signal receiver and the acoustic wave modifier are implanted or implantable to a location of the eye of the patient. For example, the at least one signal receiver and the acoustic wave modifier can be integrated into a sensor and that sensor may be implanted or implantable to a location adjacent the eye of the patient. As an example, the at least one signal receiver and/or the acoustic wave modifier can be implanted, as part of a sensor or otherwise, into the suprachoroidal space of the eye of a patient. Such implantation is particularly desirable for making frequent determinations of the intraocular pressure of the eye. For example, the intraocular pressure of the eye can be determined, using the reader or otherwise, at least twice, at least four times, at least twenty times or even continuously in a twenty-four (24) hour period of time.

According to one exemplary aspect of the present disclosure, the surface acoustic wave modifier is comprised of at least one and typically a plurality of reflectors in communication with (e.g., disposed upon) a membrane. The membrane is then put in communication and/or contact (e.g., direct or indirect contact) with a surface of the eye where the surface of the eye moves in response to changes in intraocular pressure of the eye. The membrane is designed to be flexible such that it moves (e.g., flexes) based upon the intraocular pressure of the eye and/or the movement of a surface of the eye. It may, for example, conform to the surface of the eye. In turn, the locations and/or shape of the reflectors, by virtue of their communication or contact with the membrane, are dictated by the intraocular pressure of the eye. Upon receipt of the initial surface acoustic wave signal, the reflectors reflect and modify the initial surface acoustic wave signal to form the modified surface acoustic wave signal. The modification of the initial surface acoustic wave signal is dependent upon the location[s] and/or shape of the reflector[s], which is dependent on the intraocular pressure of the eye. This modified surface acoustic wave can then be converted to the second signal, for example by the at least one signal receiver, such that the second signal can be compared to the first signal to determine the value indicative of the intraocular pressure.

It is contemplated that the membrane could be placed in contact with the cornea or another portion of the eye. However, it has been found that placing the membrane in contact with a choroid of the eye provides greater sensitivity of the membrane and/or reflectors to intraocular pressure of the eye since the choroid tends to exhibit greater movement and/or elasticity in response to changes in the intraocular pressure of the eye. As such, implanting the surface acoustic wave modifier, either with or without the at least one signal receiver, is particularly desirable in such a configuration since it allows the membrane to contact the choroid.

As suggested, the present disclosure also includes a method of sensing intraocular pressure of an eye of a patient. The method disclosed herein can be implemented with the system disclosed herein or may be otherwise implemented with an alternative system or sensor. The method includes: receiving a first signal with the at least one signal receiver, the at least one signal receiver producing an initial surface acoustic wave signal based upon the first signal; transmitting the initial surface acoustic wave signal to the surface acoustic wave modifier; modifying the initial surface acoustic wave signal using the surface acoustic wave modifier to produce a modified surface acoustic wave signal based on the intraocular pressure of the eye of the patient; transmitting the modified surface acoustic wave signal to the at least one signal receiver; and producing a second signal based upon the modified surface acoustic wave signal using the at least one signal receiver, the second signal being comparable to the first signal to determine a value indicative of the intraocular pressure of the eye. It is contemplated that the method may be carried out at multiple locations of the eye. However, according to one particularly desirable aspect, the method includes implanting a sensor into a suprachoroidal space of the eye of the patient, the sensor including the surface acoustic wave modifier and the at least one signal receiver. It is also contemplated that the method can include comparing the first signal to the second signal with a signal reader for determining the value indicative of the intraocular pressure of the eye. Moreover, the value indicative of the intraocular pressure of the eye can be determined, using the signal reader or otherwise, at least twice, at least four times, at least twenty times or even continuously in a twenty-four (24) hour period of time.

Within the methodology, various characteristics of the system may be employed. The reader can be a radio frequency signal reader and/or the first signal and second signal can be radio frequency signals. The signal reader can be a stand-alone dedicated instrument, or it can be integrated into a smartphone or other device. The surface acoustic wave modifier can include reflectors disposed upon a membrane with the membrane in communication with a choroid or other surface of the eye such that movement of the choroid or other surface of the eye in response to changes in the intraocular pressure of the eye cause deformation of the membrane and movement of the reflectors. When present, the reflectors can modify the initial surface acoustic wave signal based upon locations and/or shape[s] of the reflectors as dictated by the deformation of the membrane. If desired, at least one signal receiver and the surface acoustic wave modifier are disposed within a housing with the membrane being at least part of that housing.

### Exemplary System and Implementation

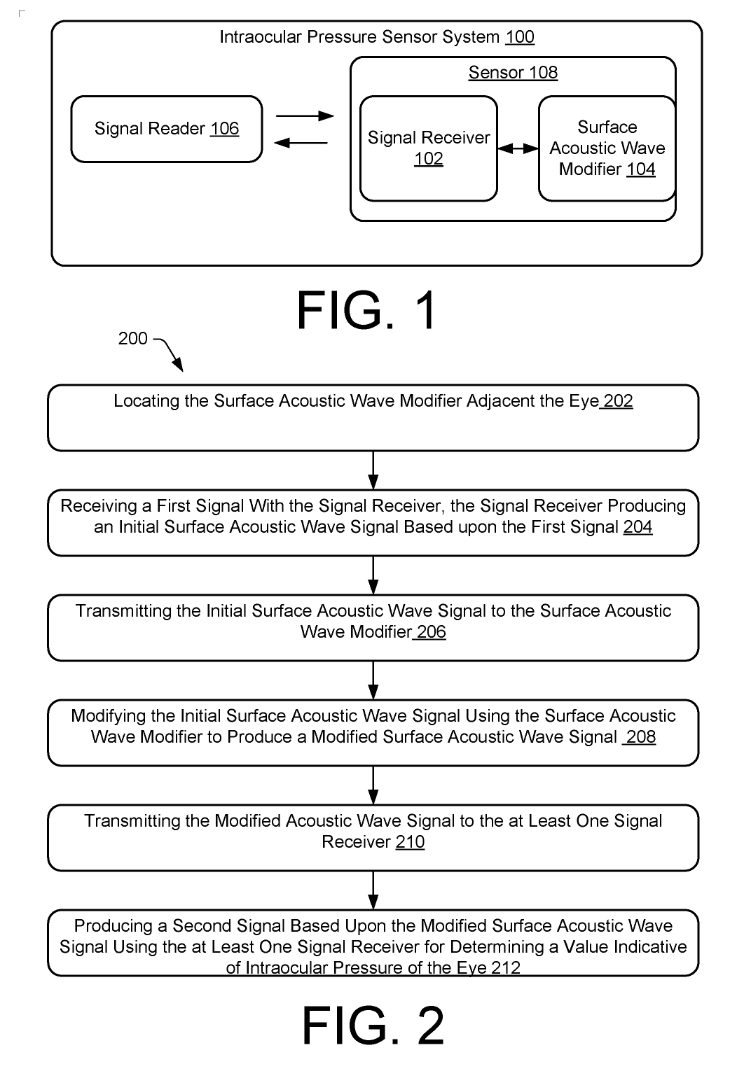

FIG. 1 provides a schematic diagram of an intraocular pressure sensor system 100 in an example implementation that is operable to measure intraocular pressure of an eye of a patient as described herein. The intraocular pressure sensor system 100 includes a signal receiver 102, a surface acoustic wave modifier 104, and a signal reader 106. In this example, the signal receiver 102 and surface acoustic wave modifier 104 are integrated together to form a sensor 108 and the sensor 108, particularly the signal receiver 102, is in signaling communication with the signal reader 106. Also, in this example, the signal receiver 102 is in signaling communication with the surface acoustic wave modifier 104. In alterative configurations, it is contemplated that the signal receiver 102, the surface acoustic wave modifier 104, and the signal reader 106 can be separate or can be selectively integrated together as needed or desired.

The signal receiver 102 has the capability of receiving signals (e.g., radio frequency signals) and converting the signals into surface acoustic wave signals. The signal receiver 102 also has the capability of receiving surface acoustic wave signals and converting them into signals (e.g., radio frequency signals) that can be read and/or interpreted by the signal reader 106. The signal receiver 102 can be, for example, a modulator (e.g., an amplitude or frequency modulator), a transducer, a combination thereof or the like. It is understood that the signal receiver 102 can be comprised of a singular component, multiple components integrated together, or multiple components that are physically separate from each other. For example, the signal receiver 102 can include a first component that converts an RF signal to a surface acoustic wave signal and a second component that converts a surface acoustic wave signal to an RF signal and those components may be separate or integrated together.

The surface acoustic wave modifier 104 has the capability to receive an initial surface acoustic wave signal from the signal receiver 102, modify that initial surface acoustic wave signal to form a modified surface acoustic wave signal, and send or transmit that modified surface acoustic wave signal. The surface acoustic wave modifier 104 also has the ability to modify the initial surface acoustic wave signal based upon the intraocular pressure of an eye. To do so, the surface acoustic wave modifier 104 is typically in communication with the eye (e.g., a surface of the eye such as a surface of the choroid or cornea of the eye) such that the surface acoustic wave modifier 104 modifies the initial surface acoustic wave signal to produce the modified surface acoustic wave signal based on the intraocular pressure of the eye.

The signal reader 106 has the capability to receive a first signal and a second signal and compare the first signal to the second signal to determine a value indicative of the intraocular pressure of the eye. It is contemplated that the signal reader 106 could be selected from a variety of different devices such as a smart phone, a computer, a tablet or the like for calculating a value indicative of intraocular pressure. Alternatively, the signal reader 106 can be a dedicated device that is designed specifically to determine the value indicative of intraocular pressure.

In one example, the signal reader 106 is a computing device that is representative of one or more computing systems and/or devices that may implement the various techniques described herein. The signal reader 106 can include a processing system, one or more computer-readable media and one or more I/O interfaces that are communicatively coupled, one to another. The processing system is representative of functionality to perform one or more operations using hardware. Accordingly, the processing system can include hardware elements that may be configured as processors, functional blocks, and so forth. This may include implementation in hardware as an application specific integrated circuit or other logic device formed using one or more semiconductors. The hardware elements are not limited by the materials from which they are formed, or the processing mechanisms employed therein. For example, processors may be comprised of semiconductor(s) and/or transistors (e.g., electronic integrated circuits (ICs)). In such a context, processor-executable instructions may be electronically executable instructions. The computer-readable storage media often includes memory/storage, which may include fixed media (e.g., RAM, ROM, a fixed hard drive, and so on) as well as removable media (e.g., flash memory, a removable hard drive, an optical disc, and so forth).

“Computer-readable storage media” may refer to media and/or devices that enable persistent and/or non-transitory storage of information in contrast to mere signal transmission, carrier waves, or signals per se. Thus, computer-readable storage media refers to non-signal bearing media.

FIG. 2 provides a flowchart 200 to help describe the operation of the intraocular pressure sensor system 100 of FIG. 1. This flowchart 200 is an example procedure. Aspects of the procedure may be implemented in hardware, firmware, or software, or a combination thereof. The procedure is shown as a set of blocks that specify operations performed by one or more devices and are not necessarily limited to the orders shown for performing the operations by the respective blocks. In at least some implementations, at least a portion of the procedure is performed by a suitably configured device, such as the signal reader 106 of FIG. 1, by executing instructions stored in a non-transitory computer-readable storage medium.

The intraocular pressure sensor system 100 is configured to sense intraocular pressure of the eye. Thus, at 202, a portion, and more specifically, the surface acoustic wave modifier 104 is placed adjacent the eye of a patient. Generally, the surface acoustic wave modifier 104 is placed adjacent a surface of the eye of the patient where that surface of the eye moves in response to changes of intraocular pressure of the eye. In turn, the manner in which the surface acoustic wave modifier 104 modifies a surface acoustic wave is dependent upon the intraocular pressure of the eye. As one example, the surface acoustic wave modifier 104 can be comprised of one or more reflectors and the locations and/or shapes of the one or more reflectors is dependent upon the intraocular pressure of the eye such that the manner in which the one or more reflectors reflect a surface acoustic wave is dependent upon the intraocular pressure of the eye.

At 204, a first signal is received by the signal receiver 102 and, based on that first signal, the signal receiver produces an initial surface acoustic wave signal. The first signal can be provided by the signal reader 106 or can be provided by a separate device altogether. It is contemplated that the first signal can be any type of signal that can be converted into a surface acoustic wave signal. For example, the first signal can be a radio frequency (RF) signal. The signal receiver 102 converts the first signal to an initial surface acoustic wave signal that is based on the first signal. For example, it is contemplated that the surface acoustic wave signal has a frequency, wavelength, or the like that is dependent upon the first signal. The first signal is also received and/or measured by the signal reader 106. For example, when the first signal is an RF signal, the signal reader 106 determines and/or measures the frequency of the first signal. If the first signal is produced by the signal reader 106, the signal reader may determine or measure a characteristic (e.g., frequency) of the first signal by producing the first signal with the particular characteristic. Alternatively, if the first signal is produced by an entity other that the signal reader 106, the signal reader 106 will have the capability to measure and/or determine that particular characteristic.

At 206, the initial surface acoustic wave signal is transmitted to the surface acoustic wave modifier 104 and, at 208, the surface acoustic wave modifier 104 modifies the initial surface acoustic wave signal to produce a modified surface acoustic wave signal. The surface acoustic wave modifier 104 converts the initial surface acoustic wave signal to a modified surface acoustic wave signal that is based on the initial surface acoustic wave signal. For example, it is contemplated that the modified surface acoustic wave signal has a wavelength, frequency, or the like that is dependent upon the initial surface acoustic wave signal. As discussed above, the surface acoustic wave modifier 104 is typically in communication with the eye (e.g., a surface of the eye such as a surface of the choroid or cornea of the eye) such that the surface acoustic wave modifier 104 modifies the initial surface acoustic wave signal to produce the modified surface acoustic wave signal based on the intraocular pressure of the eye.

At 210, the modified surface acoustic wave signal is transmitted to the signal receiver 102. At 212, a second signal is produced by the signal receiver 102 based upon the modified surface acoustic wave signal for determining the value indicative of intraocular pressure of the eye. It is contemplated that the second signal can be any type of signal resulting from conversion of the modified surface acoustic wave signal into the second signal. For example, the second signal can be a radio frequency (RF) signal. The signal receiver 102 converts the initial surface acoustic wave signal into a second signal that is based on the modified surface acoustic wave signal. For example, it is contemplated that the second signal has a frequency, wavelength, or the like that is dependent upon the modified surface acoustic wave signal. Like the first signal, the second signal is also received and/or measured by the signal reader 106. For example, when the second signal is an RF signal, the signal reader 106 determines and/or measures the frequency of the second signal.

Generally, it is contemplated that the first signal, the second signal, the initial surface acoustic wave signal and the modified surface acoustic wave signal may be transmitted and/or received directly to and/or from the signal receiver 102, the surface acoustic wave modifier 104, or the signal reader 106 where desired or appropriate. Such components may include antennas where desired to aid such communications. It is also contemplated that such signals may travel through intermediaries and may be changed or converted by such intermediaries as they travel to and/or from the respective components. As one example, it is contemplated that the first signal, the second signal or both may be converted to signals that can travel to the signal reader 106 via the world wide web. Suitable communication pathways for the various signals include, but are not limited to, direct wire, buss or other material communication protocols, RF communication protocols, Bluetooth protocols, WiFi protocols, Bluetooth low energy (BLE) protocols, Medical Device Radiocommunications Service (MedRadio) protocols, combination thereof or the like.

Once the first signal and second signal are received, determined and/or measured by the signal reader 106, the first signal can be compared to the second signal to determine a value indicative of the intraocular pressure of the eye. Generally, this is done by determining a difference between one or more values (e.g., frequency or wavelength) of the first signal and one or more values (e.g., frequency or wavelength) of the second signal and correlating that difference to the value indicative of the intraocular pressure of the eye. For example, and without limitation, the first signal can be a first radio frequency signal that is converted to the initial surface acoustic wave signal by the signal receiver 102 and that initial surface acoustic wave signal is then modified by the surface acoustic wave modifier 104 to produce the modified surface acoustic wave signal, which is then converted by signal receiver 102 to produce the second signal as a second radio frequency signal. In this example, the signal reader 106, upon receiving the first radio frequency signal and the second radio frequency signal can determine the value indicative of the intraocular pressure of the eye since the modification of the initial surface acoustic wave signal to produce the modified surface acoustic wave signal is dependent on the intraocular pressure of the eye.

The correlation between the first signal and the second signal, which is typically performed by the signal reader 106, can be performed according to a variety of protocols. As an example, empirical data can be experimentally derived and developed to understand how the intraocular pressure of the eye modifies the initial surface acoustic wave signal to arrive at the modified surface acoustic wave signal and such data can be correlated to the difference between the first signal and second signal to determine the value indicative of the intraocular pressure. As other examples, scientific publications, known sensor (e.g., SAW sensor) behavior, simulations (e.g., finite element simulations), theoretical calculations, combinations thereof or the like may be employed to provide a correlation between the first signal and second signal to the value indicative of intraocular pressure (e.g., the intraocular pressure itself).

It is contemplated that the value indicative of intraocular pressure can be provided simply as a difference between the first signal and the second signal. However, it is also contemplated that the difference between the first signal and the second signal can be correlated to a value of intraocular pressure of the eye, which may be provided in pressure units such as millimeters mercury (i.e., mmHg).

### Exemplary Implementation

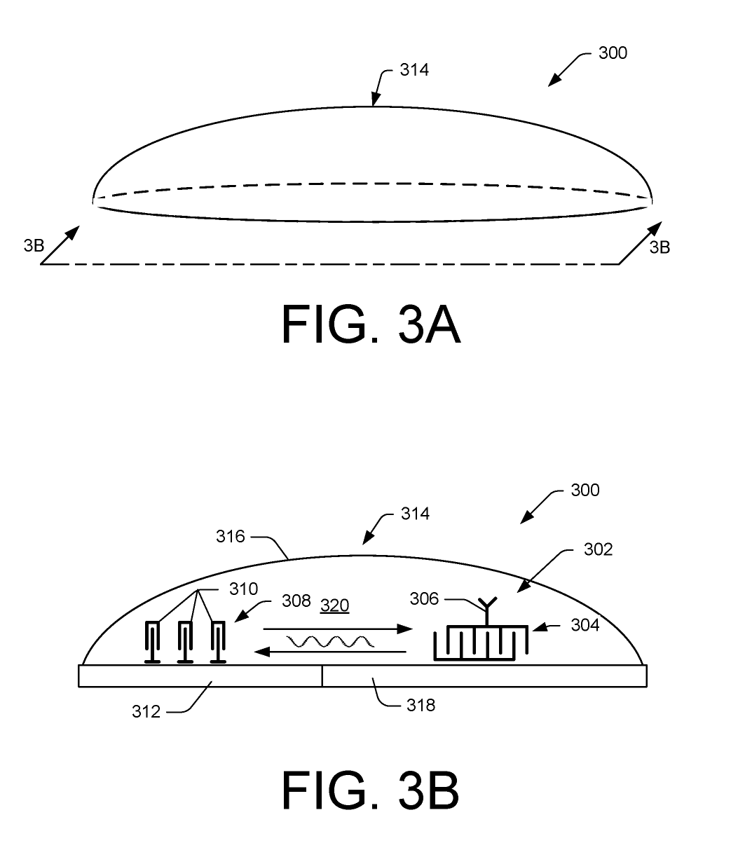

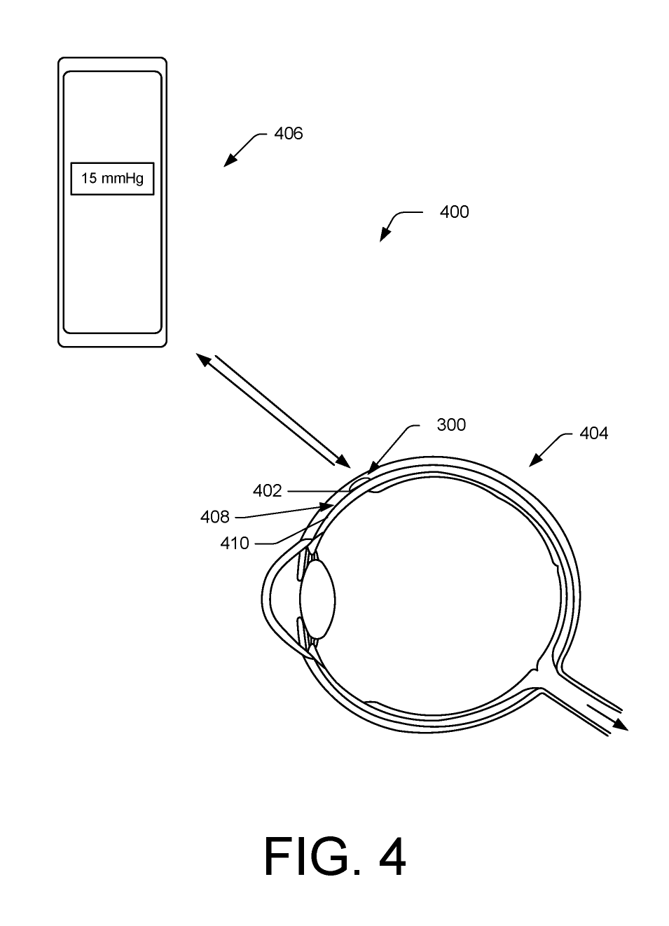

With reference to FIGS. 3A, 3B and 4, a specific example of a sensor 300 and sensor system 400 in accordance with an aspect of the present disclosure are shown. This example sensor 300 and sensor system 400 are not limiting unless otherwise specifically recited.

The sensor 300 includes a signal receiver 302, which is comprised of a transducer 304 (e.g., an interdigital transducer) and an antenna 306. The sensor 300 also includes a surface acoustic wave modifier 308, which is comprised of a plurality of reflectors 310 disposed upon a membrane 312. The signal receiver 302 and the surface acoustic wave modifier 308 are disposed within a housing 314 of the sensor 300. As can be seen, the housing 314 includes a generally arcuate wall 316 and a generally planar wall 318 defining an internal space 320 that is enclosed and/or sealed to house the plurality of reflectors 310 and the signal receiver 302, including both the transducer 304 and the antenna 306. In this instance, the membrane 312 is being considered part of the housing 314 and, particularly, the generally planar wall 318 of the housing 314. However, it is contemplated that the membrane 312 can be considered as a part of the surface acoustic wave modifier 308 such that the housing 314 and the membrane 312 of the surface acoustic wave modifier 308 cooperatively enclose and/or seal the plurality of reflectors 310 and/or the signal receiver 302 therein.

The non-membrane portion of the housing 314 and the membrane 312 may be formed of a variety of materials and may be formed of the same or separate materials. The non-membrane portion of the housing 314 is typically formed of a relatively rigid material that will generally maintain the shape and/or volume of the space 320 within housing 314 relatively consistent. Examples of suitable materials for forming the non-membrane portion of the housing 314 include, without limitation, high density polyethylene (HDPE), parylene, silicon, ceramics, acrylics, polyurethane, combinations thereof or the like. The membrane 312 is typically substantially more flexible than the non-membrane portion of the housing 314. Examples of suitable materials for forming the membrane 312 include, without limitation, elastomer, silicone, low density polyethylene (LDPE), combinations thereof or the like. Various different molding or shaping techniques such as injection molding, etching, milling, grinding or the like may be employed to shape and/or form both the non-membrane portion of the housing 314 and the membrane 312. According to one non-limiting aspect of this disclosure, the membrane 312 and the non-membrane portion of the housing 314 are co-molded together. Alternatively, the membrane 312 and non-membrane portion of the housing 314 can be separately shaped or molded and then assembled together, for example, using adhesive.As another example, the membrane 312 may be formed by etching, milling, grinding or the like to create the membrane 312 as a relatively thin and flexible substrate. As yet another example, an encapsulation (not shown) may be layered on a portion or the entirety of the housing and may form a portion or the entirety of the membrane 312.It is also contemplated that the space 320 may be empty or may be filled with a material for assisting in maintaining the integrity of the housing 314.

The sensor system 400 of FIG. 4 includes the sensor 300 of FIGS. 3A and 3B implanted into a suprachoroidal space 402 of the eye 404 of the patient. The sensor system 400 also includes a signal reader 406 illustrated as a smart phone, but which can be any of a number of devices as discussed herein.

The pressure sensor system 400 is configured to sense intraocular pressure of the eye 404. The sensor 300 is implanted in the suprachoroidal space 402 such that the membrane 312 contacts or is at least in physical communication with a surface 408 of a choroid 410 of the eye 404 such that the location (e.g., bending or curvature) of the membrane 312 is, at least in part, dictated by the movement, location of the choroid 410 and/or the transmission of intraocular pressure by the choroid 410 to the membrane 312. The choroid 410 of the eye 404 is known to move and/or transmit the intraocular pressure of the eye 404 and, thus, the location[s] (e.g., bending or curvature) and/or shape[s] of the membrane 312 and/or the reflectors 310 is dependent upon the location of choroid 410 and/or transmission of the intraocular pressure by the choroid 410 of the eye 404 to the membrane 312 and/or the reflectors 310. In turn, the manner in which the surface acoustic wave modifier 308 modifies a surface acoustic wave is dependent upon the intraocular pressure of the eye. In particular, the reflectors 310 and the location of the reflectors 310 is dependent upon the intraocular pressure of the eye such that the way the reflectors 310 reflect a surface acoustic wave is dependent upon the intraocular pressure of the eye.

The sensor 300 can be implanted in the suprachoroidal space 402 using a variety of techniques. For example, a surgeon can make an incision, and the surgeon can use one or more surgical tools to pass the sensor 300 through the incision into the suprachoroidal space 402. As another example, an injector (e.g., a disposable or autoclavable injector) with a syringe-like tip can be used to create an opening into the suprachoroidal space 402 and then the injector can be used to inject the sensor 300 into the suprachoroidal space 402. If desired, the surgeon can use stitches or other fastening devices to help secure the sensor 300 in place within the suprachoroidal space 402.

In operation, a first RF signal is received by the transducer 304 (e.g., via the antenna 306) and, based on that first RF signal, the transducer 304 produces an initial surface acoustic wave signal. The first RF signal can be provided by the signal reader 406 or can be provided by a separate device altogether. The signal receiver 302 converts the first RF signal to an initial surface acoustic wave signal that is based on the first RF signal. The transducer 304 converts the first RF signal into a surface acoustic wave signal by employing a piezoelectric material, which generates mechanical waves upon the application of an electric field. When the first RF signal is applied to electrodes of the transducer 304, it creates alternating electric fields that induce periodic stress in the piezoelectric material. This stress leads to the generation of the initial surface acoustic wave signal, which propagates along a surface. The specific design of the transducer 304, including the arrangement and spacing of the electrodes, as well as the first RF signal determines the frequency and characteristics of the generated initial surface acoustic wave signal. The first RF signal is also received and/or measured by the signal reader 406. For example, the signal reader 406 may determines and/or measure the frequency of the first RF signal. If the first RF signal is produced by the signal reader 406, the signal reader 406 may determine or measure a characteristic (e.g., frequency) of the first RF signal by producing the first RF signal such that it has the aforementioned characteristic. Alternatively, if the first RF signal is produced by an entity other that the signal reader 406, the signal reader 406 will have the capability to measure and/or determine that characteristic.

The initial surface acoustic wave signal is transmitted to the surface acoustic wave modifier 308 and, the surface acoustic wave modifier 308 modifies the initial surface acoustic wave signal to produce a modified surface acoustic wave signal. The surface acoustic wave modifier 308 converts the initial surface acoustic wave signal to a modified surface acoustic wave signal that is based on the initial surface acoustic wave signal. For the illustrated sensor, the reflectors 310 reflect the initial surface acoustic wave and, based on the position of the reflectors 310, the reflection produces the modified surface acoustic wave. As discussed, the reflectors 310, particularly the location of the reflectors 310 is dependent upon the intraocular pressure of the eye such that the manner in which the reflectors 310 reflect a surface acoustic wave is dependent upon the intraocular pressure of the eye. Thus, the difference between the initial surface acoustic wave and the modified surface acoustic wave is indicative of the intraocular pressure of the eye 404.

The modified surface acoustic wave signal is transmitted to the transducer 304. The transducer 304 then produces a second RF signal that is based upon the modified surface acoustic wave signal. The transducer 304 converts the modified surface acoustic wave signal into the second RF signal using a piezoelectric material that responds to mechanical waves. As the modified surface acoustic wave travels along a surface of the transducer, it induces localized mechanical deformations in the piezoelectric material. These mechanical changes, in turn, create alternating electric fields, which generate a corresponding electrical signal. When the modified surface acoustic wave signal interacts with electrodes of the transducer 304, the electric field is modulated according to the characteristics of the modified surface acoustic wave signal, leading to the production of the second RF signal. Like the first RF signal, the second RF signal is also received and/or measured by the signal reader 406. For example, when the second signal is an RF signal, the signal reader 106 determines and/or measures the frequency of the second signal.

Once the first RF signal and second RF signal are received, determined and/or measured by the signal reader 406, the first RF signal can be compared to the second RF signal to determine a value indicative of the intraocular pressure of the eye 404. Generally, this is done by determining a difference between one or more values (e.g., frequency or wavelength) of the first RF signal and one or more values (e.g., frequency or wavelength) of the second RF signal and correlating that difference to the value indicative of the intraocular pressure of the eye. For example, the signal reader 406, upon receiving the first RF signal and the second RF signal can determine the value indicative of the intraocular pressure of the eye since the modification of the initial surface acoustic wave signal to produce the modified surface acoustic wave signal is dependent on the intraocular pressure of the eye.

The correlation between the first RF signal and the second RF signal, which is typically performed by the signal reader 406, can be performed according to a variety of protocols. As discussed, empirical data can be experimentally derived and developed to understand how the intraocular pressure of the eye of modifies the initial surface acoustic wave signal to arrive at the modified surface acoustic wave signal and such data can be correlated to the difference between the first RF signal and second RF signal to determine the value indicative of the intraocular pressure. Of course, as discussed herein, other techniques may be used as well.

It is contemplated that the value indicative of intraocular pressure can be provided simply as a difference between the first RF signal and the second RF signal. However, it is also contemplated that the difference between the first RF signal and the second RF signal can be correlated to a value of intraocular pressure of the eye, which may be provided in pressure units such as millimeters mercury (i.e., mmHg) as shown as a display on the signal reader 406.

### Exemplary Additions or Alternatives

It is contemplated that additional components can be included within the intraocular pressure sensor system. For example, a surface acoustic wave temperature sensor could be included in the sensor 108 of FIG. 1 or sensor 300 of FIGS. 3A, 3B, and 4. Such a temperature sensor could produce wave modification in a manner similar to that of the surface acoustic wave modifier 104 such that the signal reader 106 could process additional signals that travel to and/or from from the signal receiver 102 as described herein. The signal reader 106 would then be able to additionally determine the temperature at or adjacent the sensor 108.

With reference to FIGS. 3A, 3B and 4, it is contemplated that, for example, the antenna 306 of the signal receiver 302 could be located remotely from the transducer 304 (e.g., outside the eye) but be in communication with the transducer 304. For example, the antenna 306 could be disposed upon the sclera of the eye so that it moves in conjunction with movements of the eye or could also be disposed on the face of a patient. In such alternatives, it may be possible to reduce the size of the overall sensor 300 and/or improve signal transmission and reception.

Again, with reference to FIGS. 3A, 3B and 4, it is contemplated that a tube could be extended from outside an eye of a patient to the backside of the membrane 312. This would provide atmospheric pressure to the backside of the membrane 312 such that sensing of intraocular pressure described herein is relative to such atmospheric pressure. Such a configuration may improve intraocular pressure measurement since the intraocular pressure would be measured relative to atmospheric pressure. Such a tube could be sealable in between measurement of intraocular pressure to reduce the risk of clogging the tube.

### Conclusion

Although the invention has been described in language specific to structural features and/or methodological acts, it is to be understood that the invention defined in the appended claims is not necessarily limited to the specific features or acts described. Rather, the specific features and acts are disclosed as example forms of implementing the claimed invention.

What is claimed is:

1. An intraocular pressure sensor system comprising:
a surface acoustic wave modifier that modifies an initial surface acoustic wave signal based on intraocular pressure of an eye of a patient to produce a modified surface acoustic wave; and
at least one signal receiver that:
i. receives a first signal and produces the initial surface acoustic wave signal based upon the first signal;
ii. transmits the initial surface acoustic wave signal to the surface acoustic wave modifier;
iii. receives the modified surface acoustic wave signal; and
iv. produces a second signal based upon the modified surface acoustic wave signal, the second signal being comparable to the first signal to determine a value indicative of the intraocular pressure of the eye.

2. The intraocular pressure sensor system of claim 1, further comprising a reader that compares the first signal to the second signal for determining the value indicative of the intraocular pressure of the eye.

3. The intraocular pressure sensor system of claim 2, wherein the reader determines the value indicative of the intraocular pressure of the eye at least twice in a period of 24 hours.

4. The intraocular pressure sensor system of claim 2, wherein the reader is a radio frequency reader, and the first signal and second signal are radio frequency signals.

5. The intraocular pressure sensor system of any one of claims claim 2, wherein the reader is integrated into a smartphone.

6. The intraocular pressure sensor system of any one of claims claim 1, wherein the at least one signal receiver includes an antenna, an interdigital transducer, or both.

7. The intraocular pressure sensor system of any one of claims claim 1, wherein the surface acoustic wave modifier includes reflectors disposed upon a membrane, the membrane being in communication with a choroid of the eye such that intraocular pressure of the eye causes deformation of the membrane and movement of the reflectors.

8. The intraocular pressure sensor system of claim 7, wherein the reflectors modify the initial surface acoustic wave signal based upon locations or shapes of the reflectors as dictated by the deformation of the membrane.

9. The intraocular pressure sensor system of claim 7, wherein the at least one signal receiver and the surface acoustic wave modifier are disposed within a housing and the membrane is at least part of that housing.

10. The intraocular pressure sensor system of any one of claims claim 1, wherein the surface acoustic wave modifier and the at least one signal receiver are disposed within a suprachoroidal space of the eye.

11. A method of sensing intraocular pressure of an eye of a patient, the method comprising:
implanting a sensor into a suprachoroidal space of the eye of the patient, the sensor including a surface acoustic wave modifier and at least one signal receiver;
receiving a first signal with the at least one signal receiver, the at least one signal receiver producing an initial surface acoustic wave signal based upon the first signal;
transmitting the initial surface acoustic wave signal to the surface acoustic wave modifier;
modifying the initial surface acoustic wave signal using the surface acoustic wave modifier to produce a modified surface acoustic wave signal based on the intraocular pressure of the eye of the patient;
transmitting the modified surface acoustic wave signal to the at least one signal receiver; and
producing a second signal based upon the modified surface acoustic wave signal using the at least one signal receiver, the second signal being comparable to the first signal to determine a value indicative of the intraocular pressure of the eye.

12. The method of sensing intraocular pressure of claim 11, further comprising comparing the first signal to the second signal with a reader for determining the value indicative of the intraocular pressure of the eye.

13. The method of sensing intraocular pressure of claim 12, wherein the reader determines the value indicative of the intraocular pressure of the eye at least twice in a period of 24 hours.

14. The method of sensing intraocular pressure intraocular pressure of claim 12, wherein the reader is a radio frequency reader, and the first signal and second signal are radio frequency signals.

15. The method of sensing intraocular pressure intraocular pressure of claim 12, wherein the reader is integrated into a smartphone.

16. The method of sensing intraocular pressure intraocular pressure of any one of claims claim 11, wherein the at least one signal receiver includes an antenna, an interdigital transducer, or both.

17. The method of sensing intraocular pressure of any one of claims claim 11, wherein the surface acoustic wave modifier includes reflectors disposed upon a membrane, the membrane being in communication with a choroid of the eye such that the intraocular pressure of the eye causes deformation of the membrane and movement of the reflectors.

18. The method of sensing intraocular pressure of claim 17, wherein the reflectors modify the initial surface acoustic wave signal based upon locations or shapes of the reflectors as dictated by the deformation of the membrane and wherein the at least one signal receiver and the surface acoustic wave modifier are disposed within a housing and the membrane is at least part of that housing.

19. An intraocular pressure sensor system comprising:
a surface acoustic wave modifier that modifies an initial surface acoustic wave signal based on intraocular pressure of an eye of a patient to produce a modified surface acoustic wave, the surface acoustic wave modifier including reflectors disposed upon a membrane, the membrane being in communication with a choroid of the eye such that changes in the intraocular pressure of the eye causes deformation of the membrane and movement of the reflectors, the reflectors modifying the initial surface acoustic wave signal based upon locations of the reflectors as dictated by the deformation of the membrane;
at least one signal receiver comprised of an antenna, an interdigital transducer, or both, the at least one signal receiver configured to:
i. receive a first radio frequency signal and produce the initial surface acoustic wave signal based upon the first radio frequency signal;
ii. transmit the initial surface acoustic wave signal to the surface acoustic wave modifier;
iii. receive the modified surface acoustic wave signal; and
iv. produce a second radio frequency signal based upon the modified surface acoustic wave signal, the second radio frequency signal being comparable to the first radio frequency signal to determine a value indicative of the intraocular pressure of the eye; and
a radio frequency reader that compares the first radio frequency signal to the second radio frequency signal for determining the value indicative of the intraocular pressure of the eye.

20. The system of claim 19, wherein the surface acoustic wave modifier and the at least one signal receiver are disposed within a suprachoroidal space of the eye.
